(19) United States
(12) Patent Application Publication
(10) Pub. No.: US 2006/0264418 A1
(43) Pub. Date: Nov. 23, 2006

(54) 6-(2,2,2-Trifluoroethylamino-7-chloro-2,3,4,5-tetrahydro-1h-benzo[d] azephine as a 5-ht2c receptor agonist

(76) Inventors: Christopher Stanley Galka, Carmel, IN (US); Michael John Rodriguez, Indianapolis, IN (US)

Correspondence Address: ELI LILLY & COMPANY, PATENT DIVISION, P.O. BOX 6288, INDIANAPOLIS, IN, 46206-6288, US

(21) Appl. No.: 10/568,136

(22) PCT Filed: Jul. 30, 2004
(86) PCT No.: PCT/US04/22299

§ 371 Date: Feb. 9, 2006

Related U.S. Application Data
(60) Provisional application No. 60/494,208, filed on Aug. 11, 2003.

(51) Int. Cl. A61K 31/55; C07D 223/16

(52) U.S. Cl. 514/217.01; 540/594

(57) ABSTRACT

The present invention provides a 7-chloro-6-(2,2,2-trifluoroethylamino)-2,3,4,5-tetrahydro-1H-benzo[d]azepine of formula (I): or a pharmaceutically acceptable salt thereof, and its use as a selective 5-HT_{2c} agonist for the treatment of 5-HT_{2c} associated disorders including obesity, obsessive/compulsive disorder, anxiety, and depression.

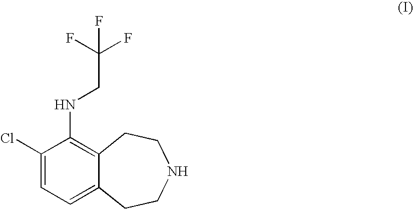

The neurotransmitter serotonin (5-hydroxytryptamine, 5-HT) has a rich pharmacology arising from a heterogeneous population of at least seven receptor classes. The serotonin 5-HT₂ class is further subdivided into at least three subtypes, designated 5-HT_{2A}, 5-HT_{2B}, and 5-HT_{2C}. The 5-HT_{2C} receptor has been isolated and characterized (Julius, et al., U.S. Pat. No. 4,985,352), and transgenic mice lacking the 5-HT_{2C} receptor have been reported to exhibit seizures and an eating disorder resulting in increased consumption of food (Julius et al., U.S. Pat. No. 5,698,766). The 5-HT_{2C} receptor has also been linked to various other neurological disorders including obesity (Vickers et al., Psychopharmacology, 167: 274-280 (2003)), hyperphagia (Tecott et al., Nature, 374: 542-546 (1995)), obsessive compulsive disorder (Martin et al., Pharmacol. Biochem. Behav., 71:615 (2002); Chou-Green et al., Physiology & Behavior, 78: 641-9 (2003)), depression (Leysen, Kelder, Trends in Drug Research II, 29: 49-61 (1998)), anxiety (Curr. Opin. Invest. Drugs 2(4), p. 317 (1993)), substance abuse, sleep disorder (Frank et al., Neuropsychopharmacology 27: 869-873 (2002), hot flashes (EP 1213017 A2), epilepsy (Upton et al., Eur. J. Pharmacol., 359: 33 (1998); Fitzgerald, Ennis, Annual Reports in Medicinal Chemistry, 37: 21-30 (2002)), and hypogonadism (Curr. Opin. Invest. Drugs 2(4), p. 317 (1993)).

Certain substituted 2,3,4,5-tetrahydro-1H-benzo[d]azepine compounds have been disclosed as useful therapeutics as for example:

U.S. Pat. No. 4,265,890 describes certain substituted 2,3,4,5-tetrahydro-1H-benzo[d]azepine compounds as dopaminergic receptor antagonists for use as antipsychotics and antiemetics, inter alia.

EP 0 285 287 describes certain substituted 2,3,4,5-tetrahydro-1H-benzo[d]azepine compounds for use as agents to treat gastrointestinal motility disorders, inter alia.

WO 93/03015 and WO 93/04686 describe certain substituted 2,3,4,5-tetrahydro-1H-benzo[d]azepine compounds as alpha-adrenergic receptor antagonists for use as agents to treat hypertension and cardiovascular diseases in which changes in vascular resistance are desirable, inter alia.

WO 02/074746 A1 describes certain substituted 2,3,4,5-tetrahydro-1H-benzo[d]azepine compounds as 5-HT_{2C} agonists for the treatment of hypogonadism, obesity, hyperphagia, anxiety, depression, sleep disorder, inter alia.

WO 03/006466 A1 describes certain substituted tricyclic hexahydroazepinoindole and indoline compounds as 5-HT ligands and consequently their usefulness for treating diseases wherein modulation of 5-HT activity is desired.

High affinity 5-HT_{2C} receptor agonists would provide useful therapeutics for the treatment of the above mentioned 5-HT_{2C} receptor-associated disorders including obesity, hyperphagia, obsessive/compulsive disorder, depression, anxiety, substance abuse, sleep disorder, hot flashes, and hypogonadism. High affinity 5-HT_{2C} receptor agonists that are also selective for the 5-HT_{2C} receptor, would provide such therapeutic benefit without the undesirable adverse events associated with current therapies. Achieving selectivity for the 5-HT_{2C} receptor, particularly as against the 5-HT_{2A} and 5-HT_{2B} receptors, has proven difficult in designing 5-HT_{2C} agonists. 5-HT_{2A} receptor agonists have been associated with problematic hallucinogenic adverse events. (Nelson et al., Naunyn-Schmiedeberg's Arch. Pharm., 359: 1-6 (1999)) 5-HT_{2B} receptor agonists have been associated with cardiovascular related adverse events, such as valvulopathy. (V. Setola et al., Mol. Pharmacology, 63:1223-1229 (2003), and ref. cited therein.)

Previous references to substituted 2,3,4,5-tetrahydro-1H-benzo[d]azepine compounds as potential therapeutics have predominately recited their uses as alpha adrenergic and/or dopaminergic modulators. Adrenergic modulators are often associated with the treatment of cardiovascular diseases (Frishman, Kotob, Journal of Clinical Pharmacology, 39: 7-16 (1999)). Dopaminergic receptors are primary targets in the treatment of schizophrenia and Parkinson's disease (Seeman, Van Tol, Trends in Pharmacological Sciences, 15: 264-270 (1994)). It will be appreciated by those skilled in the art that selectivity as against these and other physiologically important receptors will generally also be preferred characteristics for therapeutics for the specific treatment of 5-HT_{2C} associated disorders as described above.

The present invention provides a compound of formula I: 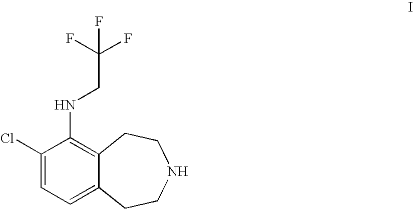
or a pharmaceutically acceptable salt thereof. This invention also provides pharmaceutical compositions which comprise a compound of formula I, or a pharmaceutically acceptable salt thereof, in association with a pharmaceutically acceptable carrier, diluent, or excipient.

In another aspect of the present invention, there is provided a method for increasing activation of the 5-HT_{2C} receptor in mammals comprising administering to a mammal in need of such activation an effective amount of a compound of formula I, or a pharmaceutically acceptable salt thereof.

The present invention also provides a method for treating obesity in mammals comprising administering to a mammal in need of such treatment an effective amount of a compound of formula I, or a pharmaceutically acceptable salt thereof. The present invention also provides a method for treating obsessive/compulsive disorder in mammals comprising administering to a mammal in need of such treatment an effective amount of a compound of formula I or a pharmaceutically acceptable salt thereof.

Furthermore, the present invention provides a method for treating depression in mammals comprising administering to a mammal in need of such treatment an effective amount of a compound of formula I or a pharmaceutically acceptable salt thereof.

Furthermore, the present invention provides a method for treating anxiety in mammals comprising administering to a mammal in need of such treatment an effective amount of a compound of formula I or a pharmaceutically acceptable salt thereof.

In preferred embodiments of the above methods of treatment utilizing a compound of formula I, or a pharmaceutically acceptable salt thereof, the mammal is a human.

In another aspect of the present invention, there is provided a compound of formula I for use in selectively increasing activation of the 5-HT_{2C} receptor and/or for use in treating a variety of disorders associated with decreased activation of the 5-HT_{2C} receptor. Preferred embodiments of this aspect of the invention include a compound of formula I for use in the treatment of obesity, hyperphagia; obsessive/compulsive disorder, depression, anxiety, substance abuse, sleep disorder, hot flashes, and/or hypogonadism. Particularly preferred embodiments of this aspect of the invention include the treatment of obesity, obsessive/compulsive disorder, depression, and/or anxiety.

In another aspect of the present invention, there is provided the use of a compound of formula I in the manufacture of a medicament for the activation of 5-HT_{2C} receptors in a mammal. In preferred embodiments of this aspect of the invention, there is provided the use of a compound of formula I in the manufacture of a medicament for the treatment of obesity, hyperphagia, obsessive/compulsive disorder, depression, anxiety, substance abuse, sleep disorder, hot flashes, and/or hypogonadism. Particularly preferred embodiments of this aspect of the invention include the use of a compound of formula I in the manufacture of medicaments for the treatment of obesity, obsessive/compulsive disorder, depression, and/or anxiety.

Additionally, the present invention provides a pharmaceutical formulation adapted for the treatment of obesity, or for the treatment of obsessive/compulsive disorder, or for the treatment of depression, or for the treatment of anxiety, each of which comprise a compound of Formula I in association with a pharmaceutically acceptable carrier, diluent or excipient.

In those instances where the disorders which can be treated by 5-HT_{2C} agonists are known by established and accepted classifications, their classifications can be found in various sources. For example, at present, the fourth edition of the Diagnostic and Statistical Manual of Mental Disorders (DSM-IV™) (I 994, American Psychiatric Association, Washington, D.C.), provides a diagnostic tool for identifying many of the disorders described herein. Also, the International Classification of Diseases, Tenth Revision (ICD-10), provides classifications for many of the disorders described herein. The skilled artisan will recognize that there are alternative nomenclatures, nosologies, and classification systems for disorders described herein, including those as described in the DSM-IV and ICD-10, and that terminology and classification systems evolve with medical scientific progress.

The term “amino protecting group” as used in this specification refers to a substituent commonly employed to block or protect the amino functionality while reacting other functional groups on the compound. Examples of such amino protecting groups include the formyl group, the trityl group, the acetyl group, the trichloroacetyl group, the trifluoroacetyl group, the chloroacetyl, bromoacetyl, and iodoacetyl groups, carbamoyl-type blocking groups such as benzyloxycarbonyl, 9-fluorenylmethoxycarbonyl (“FMOC”), t-butoxycarbonyl (t-BOC), and like amino protecting groups. The species of amino protecting group employed is not critical so long as the derivatized amino group is stable to the conditions of subsequent reactions on other positions of the molecule and can be removed at the appropriate point without disrupting the remainder of the molecule. The selection and use (addition and subsequent removal) of amino protecting groups is well known within the ordinary skill of the art. Fuer examples of groups referred to by the above terms are described by T. W. Greene and P. G. M. Wuts, “Protective Groups in Organic Synthesis”, 3^{rd} edition, John Wiley and Sons, New York, N.Y., 1999, chapter 7, hereafter referred to as “Greene”.

The term “pharmaceutical” or “pharmaceutically acceptable” when used herein as an adjective, means substantially non-toxic and substantially non-deleterious to the recipient.

By “pharmaceutical composition” it is further meant that the carrier, solvent, excipients and salt must be compatible with the active ingredient of the composition (e.g. a compound of formula I). It is understood by those of ordinary skill in this art that the terms “pharmaceutical formulation” and “pharmaceutical composition” are generally interchangeable, and they are so used for the purposes of this application.

It is generally understood by those skilled in this art, that compounds intended for use in pharmaceutical compositions are routinely, though not necessarily, converted to a salt form in efforts to optimize such characteristics as the handling properties, stability, pharmacokinetic, and/or bioavailability, etc. Methods for converting a compound to a given salt form are well known in the art (see for example, Berge, S. M, Bighley, L. D., and Monkhouse, D. C., J. Pharm. Sci., 66:1, (1977)). In that the compound of the present invention is an amine and therefore basic in nature, it readily reacts with a wide variety of pharmaceutically acceptable organic and inorganic acids to form pharmaceutically acceptable acid addition salts therewith. Such salts are also embodiments of this invention.

Typical inorganic acids used to form such salts include hydrochloric, hydrobromic, hydroiodic, nitric, sulfuric, phosphoric, hypophosphoric, metaphosphoric, pyrophosphoric acid, and the like. Salts derived from organic acids, such as aliphatic mono and dicarboxylic acids, phenyl substituted alkanoic acids, hydroxyalkanoic and hydroxyalkandioic acids, aromatic acids, aliphatic and aromatic sulfonic acids, may also be used. Such pharmaceutically acceptable salts thus include chloride, bromide, iodide, nitrate, acetate, phenyl acetate, trifluoroacetate, acryl ate, ascorbate, benzoate, chlorobenzoate, dinitrobenzoate, hydroxybenzoate, methoxybenzoate, methylbenzoate, o-acetoxybenzoate, isobutyrate, phenylbutyrate, α-hydroxybutyrate, butyne-1,4-dicarboxylate, hexyne-1,4-dicarboxylate, caprate, caprylate, cinnamate, citrate, formate, fumarate, glycollate, heptanoate, hippurate, lactate, malate, maleate, hydroxymaleate, malonate, mandelate, nicotinate, isonicotinate, oxalate, phthalate, teraphthalate, propiolate, propionate, phenylpropionate, salicylate, sebacate, succinate, suberate, benzenesulfonate, p-bromobenzenesulfonate, chlorobenzenesulfonate, ethylsulfonate, 2-hydroxyethylsulfonate, methylsulfonate (mesylate), naphthalene-1-sulfonate, naphthalene-2-sulfonate, naphthalene-1,5-sulfonate, p-toluenesulfonate, xylenesulfonate, tartrate, and the like.

It is well known that such compounds can form salts in various molar ratios to provide for example the hemi-acid, mono-acid, di-acid salts, etc.

The term “effective amount” means an amount of a compound of formula I which is capable of activating 5-HT_{2C} receptors and/or elicit a given pharmacological effect. The term “suitable solvent” refers to any solvent, or mixture of solvents, inert to the ongoing reaction that sufficiently solubilizes the reactants to afford a medium within which to effect the desired reaction.

The following terms and abbreviations are used herein:

“2B-3 ethanol” means ethanol denatured with toluene.

“Anal. Calc'd” means calculated elemental analysis.

“BINAP” means 2,2′-bis(diphenylphosphino)-1,1′binaphthyl.

“bp” means boiling point.

“CV” means calorific value of oxygen.

“DCM” means dichloromethane (i.e. methylene chloride, CH₂Cl₂).

“DMF means N,N-dimethylformamide.

“DMSO” means dimethylsulfoxide (i.e. methyl sulfoxide).

“DOI” means (±)-1-(2,5-dimethoxy-4-[¹²⁵I]-iodophenyl)-2-aminopropane.

“EE” means energy expenditure.

“EDTA” means ethylenediaminetetraacetic acid.

“GDP” means guanosine diphosphate.

“GTP” means guanosine triphosphate.

“GTPγ[³⁵S]” means guanosine triphosphate having the terminal phosphate substituted with ³⁵S in place of an oxygen.

“ISPA” means immunoadsorption scintillation proximity assay.

“mp” means melting point.

“MS (ES+)” means mass spectroscopy using electrospray ionization.

“MTBE” means methyl t-butyl ether.

“NBS” means N-bromosuccinimide.

“NMR” means nuclear magnetic resonance.

“Pd(OAc)₂” means palladium (II) acetate ((CH₃CO₂)₂Pd).

“Pd(PPh₃)₄” means tetrakis(triphenylphosphine)palladium(0).

“Pd₂(dba)₃” means tris(dibenzylideneacetone)dipalladium(0).

“RQ” means respiratory quotient.

“Sudan III” means 1-((4-phenylazo)phenylazo)-2-naphthalenol.

“Tf” in a chemical structure means the trifluoromethylsulfonyl moiety (—SO₂CF₃).

“TFA” means trifluoroacetic acid.

“TFAA” means trifluoroacetic anhydride.

“Tf₂O” means trifluomethanesulfonic anhydride. “TLC” means thin layer chromatography.

“p-TsOH.H₂O” means para-toluenesulfonic acid mono-hydrate.

The compound of the present invention and its salts may be synthesized from N-protected 6-hydroxy-2,3,4,5-tetrahydro-1H-benzo[d]azepine by chlorination at the 7-position, followed by introduction of the fluoroethylamino group at the 6-position via an appropriately reactive intermediate, such as a trifluoromethylsulfonic acid ester. This coupling product is then deprotected to obtain the free base and optionally converted to a salt as desired. (See Scheme I and Examples 1-3)

The N-protected 6-hydroxy-2,3,4,5-tetrahydro-1H-benzo[d]azepine can be obtained from 5-hydroxy-1,4-dihydronaphthalene via protection of the hydroxy group, cleavage of the double bond, as for example by ozonolysis, reductive work-up to yield the diol, conversion of the diol to a di-sulfonic acid ester, followed by reaction with ammonia to effect amination and ring closure, and consequent protection of the amino group, and finally deprotection of the 6-hydroxy group (see Scheme I and Example 1).

Suitable reaction conditions for the individual steps in this scheme are well known in the art and appropriate substitutions of solvents and co-reagents are within the skill of the art. Likewise, it will be appreciated by those skilled in the art that synthetic intermediates may by isolated and/or purified by various well known techniques as needed or desired, and that frequently, it will be possible to use various intermediates directly in subsequent synthetic steps with little or no purification. It will also be appreciated that alternative routes of synthesis for the present inventive compound and its salts are within the skill of the art, using well known methods.

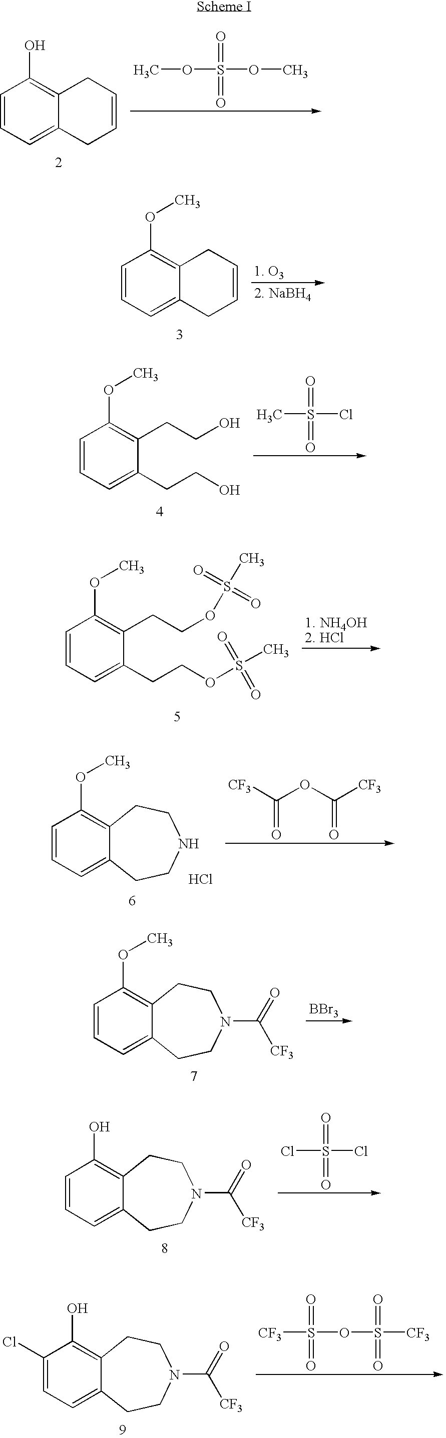

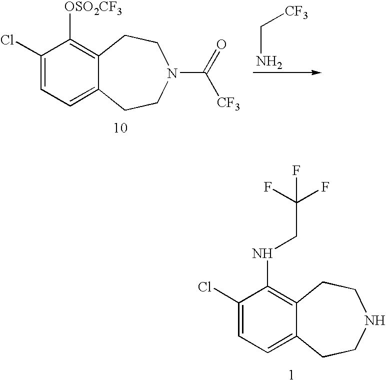

### EXAMPLE 1

### 7-Chloro-6-(2,2,2-trifluoroethylamino)-2,3,4,5-tetrahydro-1H-benzo[d]azepine-free base

### 5-Methoxy-1,4-dihydronaphthalene [3]

Add powdered potassium carbonate (193.1 g, 1.397 mol) to a solution of 5-hydroxy-1,4-dihydronaphthalene [2] (68.08 g, 90% potency based on ¹H-NMR, 0.4657 mol, from Societa Italiana Medicinala Scandicci, s.r.l., Reggello (Firenze), Italy) in ethanol (700 mL). Cool the solution to 0° C. with ice/water and add dimethyl sulfate, (88.1 g, 66.1 mL, 0.699 mol) dropwise, maintaining the temperature between 5° C. and 10° C. Then heat the reaction mixture to 40° C. until the TLC (10:1 hexane:ethyl acetate) shows the absence of starting material (about 2 hr.). Filter off the solids by vacuum filtration and remove the solvent in vacuo. Dilute the residual brown oil with diethyl ether (500 mL), wash with 10% aqueous NH₄OH (500 mL), water (500 mL), saturated aqueous NaCl (500 mL), dry the organic layer over Na₂SO₄, filter and concentrate in vacuo to give the crude product as a brown oil (73 g). Purify the crude product by short path distillation under vacuum (bp 120-130° C./5 Torr) to give the title compound as a clear oil (69.0 g, 92.5% potency corrected)(contains some 1,2,3,4-tetrahydro-5-methoxynaphthalene as an impurity). ¹H NMR (300 MHz, CDCl₃), δ 7.15 (t, 1H, J=7.9), 6.72 (dd, 2H, J=15.7, 7.9), 5.93-5.88 (m, 2H), 3.83 (s, 3H), 3.42-3.39 (m, 2H), 3.30-3.28 (m, 2H); R_{f}=0.58 eluting with 10:1 hexane:ethyl acetate.

### 2,3-Bis-(2-hydroxyethyl)-1-methoxybenzene [4]

Charge a four-neck 5 L flask equipped with an over-head mechanical stirrer, reflux condenser, thermocouple, and gas dispersion apparatus with 5-methoxy-1,4-dihydronaphthalene [3] (264.54 g, 89.5% potency based on ¹H-NMR, 1.478 mol) in DCM (1.3 L) and 2B-3 ethanol (1 L). Add sudan III (10 mg) to give a faint red color. Cool the solution to −65° C. or lower, then pass O₃ through the solution until the solution turns a light yellow color and the TLC (10:1 hexane:ethyl acetate, KMnO₄ stain) shows the absence of the starting material (about 30 hr.). Transfer the solution via cannula into a slurry of NaBH₄ (97.85 g, 2.59 mol) in 2B-3 ethanol (500 mL) cooled in ice/water. It is important that the temperature be maintained at or above 0° C., as, for example between 0° C. and 110° C., throughout the transfer to ensure the ozonide is completely reduced to the diol. After the transfer is complete, warm the solution to ambient temperature and stir for about 30 min. Cool the slurry to 0° C. with ice/water then slowly add acetone (540 mL, 7.4 mol) to remove excess NaBH₄. After all the solids dissolve, remove the solvent in vacuo. Dissolve the yellow solid in DCM (1 L) and water (1 L), separate the layers and extract the aqueous layer with DCM (750 mL). Wash the combined organic layers with saturated aqueous NaCl (1.5 L), add toluene (750 mL) and remove the solvent in vacuo. Redissolve the solid in DCM (500 mL) with heating, then add toluene (750 mL) and concentrate the solution in vacuo to give the title compound as a light yellow solid (283.7 g, 89% potency corrected, mp 82-83° C.)(contains 1,2,3,4-tetrahydro-5-methoxynaphthalene as an impurity (8.6%)). Further purify the product by vacuum drying overnight at 75° C., 5 Torr, to remove all but trace amounts of the 1,2,3,4-tetrahydro-5-methoxynaphthalene impurity. ¹H NMR (300 MHz, CDCl₃), δ 7.16 (dd, 1H, J=8.2, 7.6), 6.83 (s, 1H, J=7.0), 6.76 (s, 1H, J=8.2), 3.85-3.77 (m, 7H), 3.01-2.91 (m, 4H), 2.35 (s, 2H); ¹³C NMR (300 MHz, DMSO-d₆), δ 157.5, 138.9, 126.5, 125.2, 122.0, 108.4, 62.1, 60.5, 55.3, 36.1, 29.6; IR (KBr): 3006, 2960, 2886, 2829, 1583, 1461, 1440, 1264, 1091, 1041 cm⁻¹; MS (ES+) m/z 178 (M⁺+1); Anal. Calc'd for C₁₁H₁₆O₃: C, 67.32; H, 8.22; N, 0. Found: C, 67.26, H, 8.10, N, 0.21; R_{f}=0.23 eluting with 95:5 DCM: methanol.

### 2,3-Bis-(2-methanesulfonyloxyethyl)-1-methoxybenzene [5]

To a slurry of 2,3-bis-(2-hydroxyethyl)-1-methoxybenzene [4] (50.6 g, 0.258 mol, 1 equiv.) and triethylamine (78.3 g, 0.774 mol, 3 equiv.) in DCM (500 mL) cooled to 0° C., add dropwise a solution of methanesulfonyl chloride (65.0 g, 0.567 mol, 2.2 equiv.) in DCM (100 mL) over 45 min. The addition is exothermic and the methanesulfonyl chloride is added at a rate to keep the temperature below 10° C. After the addition is complete, warm the reaction to ambient temperature. Wash the solution with water (2×500 mL), and then saturated aqueous NaCl (750 mL). Dry the organic layer over Na₂SO₄, filter and concentrate in vacuo to obtain the title compound as a dark yellow oil (87.4 g, 96.2%), which is used in the next reaction without further purification. An analytical sample is obtained by flash column chromatography eluting with 100% diethyl ether. ¹H NMR (300 MHz, CDCl₃), δ 7.20 (t, 1H, J=7.9), 6.82 (s, 1H, J=7.2), 6.80 (s, 1H, J=8.2), 4.41-4.34 (m, 4H), 3.83 (s, 3H), 3.16-3.09 (m, 4H), 2.91 (s, 3H), 2.87 (s, 3H); ¹³C NMR (300 MHz, CDCl₃), δ 158.07, 136.55, 128.26, 123.34, 122.39, 109.24, 69.88, 69.08, 55.55, 37.35, 37.14, 32.57, 26.47; ¹³C NMR (300 MHz, DMSO-d₆), δ 157.58, 136.79, 127.81, 122.91, 122.00, 109.33, 70.19, 68.88, 55.55, 36.49, 36.47, 31.56, 25.72; IR (KBr): 1586.8, 1469.4, 1358.51, 1267.3, 1173.9, 1105.4, 972.4, 954.6, 914.3 cm⁻¹; MS (ES+) m/z 257 (M⁺+1); Anal. Calc'd. for C₁₃H₂₀O₇S₂: C, 44.31; H, 5.72; N, 0. Found: C, 44.22, H, 5.68, N, 0.13;

R_{f}=0.72 eluting with 95:5 DCM: methanol.

### 6-Methoxy-2,3,4,5-tetrahydro-1H-benzo[d]azepine

Dissolve 2,3-bis-(2-methanesulfonyloxyethyl)-1-methoxybenzene [5] (474.4 g, 1.346 mol) in acetonitrile (7 L) and split the mixture into two equal lots. In two separate runs, add concentrated aqueous NH₄OH (3.5 L) and charge the solution to a pressure vessel (PARR apparatus). Heat the solution in closed reactor to 100° C. over 20 min. (internal pressure reaches about 100 psi ?), and maintain at 100° C. until the reaction is complete (about 1 hr., HPLC monitored). Cool the reaction mixture to ambient temperature. Combine the two lots and remove the solvent in vacuo. Dissolve the residue in MTBE (3.5 L) and water (3.5 L). Adjust the pH to 6.5 using 2 M NaOH or 1 M HCl as appropriate (typically the pH is about pH=5.1 and the adjustment requires about 50 mL 2 M NaOH). Discard the organic layer, adjust the aqueous layer to pH=13 using 50% NaOH (about 150 mL). Extract with MTBE (2×3.5 L), wash the combined organic layers with saturated aqueous NaCl (3.5 L), dry over Na₂SO₄, filter and concentrate in vacuo to give the title compound as a crude yellow oil that solidifies upon standing (179.3 g). Use the material in the next step without further purification. Prepare an analytical sample by purification by two Kugelrohr distillations to give a clear oil that solidifies upon standing, mp 44.3-45.0° C. ¹³C NMR (300 MHz, DMSO-d₆) δ 156.1, 144.4, 130.3, 126.2, 121.5, 108.9, 55.5, 48.2, 47.9, 39.9, 29.1; MS (ES+) m/z 163 (M⁺+1); Anal. Calc'd for C₁₁H₁₅NO: C, 74.54; H, 8.53; N, 7.90. Found: C, 74.28, H, 8.62, N, 7.86.

### 6-Methoxy-2,3,4,5-tetrahydro-1H-benzo[d]azepine Hydrochloride [6]

Dissolve crude 6-methoxy-2,3,4,5-tetrahydro-1H-benzo[d]azepine (above, 35.1 g, 0.198 mol) in 2B-3 ethanol (250 mL), heat the solution to reflux and add 2 M HCl in ethanol (108.9 mL, 0.218 mol, 1.1 equiv.). Slowly add heptane (700 mL) over 10 min., then remove the heating mantle and cool the solution to ambient temperature, and finally continue the cooling with an ice/water mixture. Collect the resulting solid by vacuum filtration and wash with cold ethanol:beptane (1:2) (3×100 mL), air-dry for 15 min. under vacuum, then further dry the product in a vacuum oven at 60° C. for 1 hr. to give the title compound as a white granular solid (35.53 g, 63%): mp 246.6-246.9° C.; ¹H NMR (300 MHz, DMSO-d₆), δ 9.82 (broad s, 1H), 7.12 (dd, 1H, J=7.6, 7.9), 6.88 (d, 1H J=8.2), 6.78 (d, 1H, J=7.3), 3.75 (s, 3H), 3.20-3.00 (m, 8H); ¹³C NMR (300 MHz, DMSO-d₆), δ 156.2, 141.3, 127.4, 127.2, 121.6, 109.7, 55.7, 44.9, 44.7, 31.6, 21.7; MS (ES+) m/z 178 (M⁺+1); Anal. Calc'd for C₁₁H₁₅ClNO: C, 62.12; H, 7.11; N, 6.59. Found: C, 61.95, H, 7.64, N, 6.58.

### 6-Methoxy-3-(2,2,2-trifluoroacetyl)-2,3,4,5-tetrahydro-1H-benzo[d]azepine [7]

To a slurry of 6-methoxy-2,3,4,5-tetrahydro-1H-benzo[d]azepine hydrochloride [6] (35.3 g, 0.165 mol, 1 equiv.) and triethylamine (69.1 mL, 0.496 mol, 3 equiv.) in DCM (300 mL) cooled to 0° C. with ice/water, add dropwise a solution of trifluoroacetic anhydride (25.7 mL, 0.182 mol, 1.1 equiv.) in DCM (40 mL) over 30 min., but at a rate that maintains the temperature below 10° C. After the addition is complete, warn the reaction mixture to ambient temperature and stir until the reaction is complete (verify by TLC using 90:10 CH₂Cl₂:methanol, about 2 hr.). Wash the solution with water (2×350 mL), and then saturated aqueous NaCl (350 mL), dry the organic layer over Na₂SO₄, filter and concentrate in vacuo to give title compound as a yellow oil that solidifies upon standing (44.9 g, 96%). Use the material without further purification in the next step. Prepare an analytical sample by flash column chromatography eluting with 40% diethyl ether in hexane, mp 74-76° C. ¹H NMR (300 MHz, CDCl₃), δ 7.16-7.11 (m, 1H), 6.81-6.74 (m, 2H), 3.81 (s, 3H), 3.79-3.64 (m, 4H), 3.11-3.07 (m, 2H), 2.99-2.95 (m, 2H); ¹H NMR (300 MHz, DMSO-d₆), δ 7.13 (dd, 1H, J=1.5, 7.0), 7.08 (d, 1H, J=1.5), 6.88-6.74 (m, 1H), 3.75 (s, 3H), 3.67-3.61 (m, 4H), 3.04-2.92 (m, 4H); ¹³C NMR (300 MHz, DMSO-d₆), δ 156.43. 156.38, 155.06, 155.00, 154.60, 154.54, 154.14, 154.08, 141.31, 141.04, 127.44, 127.18, 127.05, 127.01, 122.27, 121.94, 121.90, 118.46, 114.64, 110.80, 109.52, 109.41, 55.63, 55.61, 47.11, 47.07, 46.67, 46.63, 45.61, 45.16, 35.90, 34.65, 26.18, 24.91; Anal. Calc'd for C₁₃H₁₄F₃NO₂: C, 57.14; H, 5.16; N, 5.13. Found: C, 57.17, H, 5.27, N, 5.08.

### 6-Hydroxy-3-(2,2,2-trifluoroacetyl)-2,3,4,5-tetrahydro-1H-benzo[d]azepine [8]

To a 1 M solution of BBr₃ (1.1 L, 1.6 equiv.), cooled to 0° C. with ice water, add 6-methoxy-3-(2,2,2-trifluoroacetyl)-2,3,4,5-tetrahydro-1H-benzo[d]azepine [7] (187 g, 0.684 mol) in DCM (200 mL) over 1 hr., while maintaining the temperature between 0° C. and 10° C. Warm the reaction mixture to ambient temperature and stir until HPLC indicates completion of the reaction (about 2 hr.). Cool the solution to 0° C. and transfer it via cannula into an ice/water solution (1.2 L), thereby precipitating the product as a white solid. Add ethyl acetate (2 L) to dissolve most of the precipitate, separate the layers and concentrate the organic layer in vacuo. Extract the aqueous layer three times with ethyl acetate (2×2 L, 1×1 L). Wash the combined organic layers with water (2 L), and then saturated aqueous NaCl (2 L), dry over Na₂SO₄, filter and concentrate in vacuo to give the title compound as a light yellow solid (166.3 g, 94%). Use the product in the next step without further purification. Prepare an analytical sample by flash column chromatography eluting with 40% diethyl ether in hexane: mp 183.0-185.2° C. ¹H NMR (300 MHz, DMSO-d₆), δ 9.39 (s, 1H), 6.94-6.88 (m, 1H), 6.72-6.68 (m, 1H), 6.61-6.57 (m, 1H), 3.67-3.32 (m, 4H), 2.99-2.86 (m, 4H); ¹³C NMR (300 MHz, DMSO-d₆), δ154.50, 141.47, 141.18, 126.77, 126.64, 125.77, 125.33, 120.38, 120.32, 118.49, 114.67, 113.64, 113.47, 47.31, 47.27, 47.00, 46.96, 45.83, 45.49, 36.17, 34.93, 26.46, 25.18, 20.66, 14.00; MS (ES+) m/z 260 (M⁺+1); Anal. Calc'd. for C₁₂H₁₂F₃NO₂: C, 55.60; H, 4.67; N, 5.40. Found: C, 55.51, H, 4.71, N, 5.29.

### 7-Chloro-6-hydroxy-3-(2,2,2-trifluoroacetyl)-2,3,4,5-tetrahydro-1H-benzo[d]azepine [9]

Heat a mixture of 6-hydroxy-3-(2,2,2-trifluoroacetyl)-2,3,4,5-tetrahydro-1H-benzo[d]azepine [8] (120.0 g, 0.4629 mol) and toluene (14.4 L) to 70° C. for 45 min. until most of the starting material is dissolved. Add diisobutylamine (1.197 g, 1.62 mL, 9.26 mmol) followed by addition of sulfuryl chloride (62.48 g, 37.19 mL, 0.463 mol) in toluene (360 mL) over 20 min. Stir the reaction mixture for 50 min. and then add additional sulfuryl chloride (4.536 g, 2.70 mL, 0.0336 mol) neat and stir the reaction mixture for 15 min. at 70° C. Cool the reaction mixture to 24° C. over 30 min. and then add 1N hydrochloric acid (2.00 L). Separate the organic layer, wash with saturated sodium hydrogencarbonate (2.00 L), wash with a saturated solution of sodium chloride (2.00 L) and then dry over sodium sulfate. Filter and remove the solvent with a rotary evaporator at 70° C. until about 672.5 g remains using the minimum effective vacuum in order to maintain a vapor phase sufficient to prevent drying above the solvent line and self-seeding, thus preventing crystallization under these conditions. Using toluene heated to 70° C., transfer the light-yellow solution to a preheated (70° C.) 3-neck flask equipped with a mechanical stirrer. Lower the temperature to 58° C. over 1 hr. If available, seed the solution with crystals of 7-chloro-6-hydroxy-3-(2,2,2-trifluoroacetyl)-2,3,4,5-tetrahydro-1H-benzo[d]azepine from a prior synthesis to enhance crystallization. After 30 min., reduce the temperature further to 55° C. and observe the initiation of the crystallization process. Hold the temperature at 55° C. for 2 hr. followed by 4 hr. at 45° C., then turn off the heat allowing the mixture to slowly reach 24° C. (ambient temperature). After stirring for 8 hr. with the heat off, cool the mixture to 0° C. for 2 hr. followed by 2 hr. at −10° C. Collect the resulting dense, white, granular crystals by vacuum filtration at −10° C. Rinse the crystals twice with cold (−10° C.) toluene and vacuum dry at 50° C., 5 Torr, for 12 hr., to obtain the title compound as a white solid (120.7 g, 99.5% purity, 88.8% yield): mp 133-134° C. MS (ES+) m/z 294 (M⁺+1). Anal. Calc'd for C₁₂H₁₁ClF₃NO₂: C, 49.08; H, 3.78; N, 4.77; Cl, 12.07. Found: C, 49.01; H, 3.63; N, 4.72; Cl, 12.32.

### 7-Chloro-3-(2,2,2-trifluoroacetyl)-6-trifluoromethylsulfonyloxy-2,3,4,5-tetrahydro-1H-benzo[d]azepine [10]

Cool a solution of 7-chloro-6-hydroxy-3-(2,2,2-trifluoroacetyl)-2,3,4,5-tetrahydro-1H-benzo[d]azepine [9] (60 g, 0.204 mol), triethylamine (62.6 mL, 0.448 mol, 2.2 equiv.), and DCM (590 mL) in an ice bath and add dropwise trifluoromethanesulfonic anhydride (43.5 mL, 0.258 mol, 1.26 equiv.) over 70 min. Remove the ice bath and stir the reaction mixture for 2 hr. Wash the reaction mixture sequentially with water (500 mL), 1N HCl (500 mL), water (500 mL), and saturated aqueous NaCl (500 mL). Dry the organic layer over Na₂SO₄ and concentrate in vacuo to give the crude product as a brown solid (90 g). Dissolve the solid in toluene (200 mL) with warming. Further purify by plug filtration chromatography over silica gel (500 g) eluting sequentially with hexane (1 L), hexane:ethyl acetate (90:10, 1 L), hexane:ethyl acetate (80:20, 1 L), and hexane:ethyl acetate (70:30, 9 L). Pool the eluents and evaporate the solvent to obtain the product as a yellow tan solid (86.3 g). Dissolve the solid in ethyl acetate (86 mL) with warning and then add hexane (700 mL). If available, seed the solution with crystals of 7-chloro-3-(2,2,2-trifluoroacetyl)-6-trifluoromethylsulfonyloxy-2,3,4,5-tetrahydro-1H-benzo[d]azepine from a prior synthesis to enhance crystallization. Allow the mixture to stand at ambient temperature for 30 min. Cool the mixture at about −10° C. for 2 hr., filter, rinse the crystals with cold (−10° C.) hexane/ethyl acetate, and air-dry on the filter under vacuum to obtain title compound as a first crop of crystals (73.54 g). Concentrate the mother liquor to obtain a solid (12.7 g). Recrystallize the solid in a mixture of ethyl acetate:hexane (15 mL:121 mL) to obtain additional title compound (7.65 g, total yield: 81.19 g, 93.5%).

### 7-Chloro-3-(2,2,2-trifluoroacetyl)-6-(2,2,2-trifluoroethylamino)-2,3,4,5-tetrahydro-1H-benzo[d]azepine

Place 7-chloro-3-(2,2,2-trifluoroacetyl)-6-trifluoromethylsulfonyloxy-2,3,4,5-tetrahydro-1H-benzo[d]azepine [10] (6.68 g, 15.7 mmol), Pd(OAc)₂ (334 mg, 1.48 mmol), (rac)-2,2′-bis(diphenylphosphino)-1,1′-binaphthyl((rac)-BINAP)(1.0 g, 1.60 mmol), and Cs₂CO₃ (7.08 g, 21.7 mmol) in a N₂-purged, 475 mL high pressure flask, containing a magnetic stir bar. Add toluene (170 mL) to the mixture and degas 3-5 times by partially evacuating the flask and flushing with nitrogen. Add 2,2,2-trifluoroethylamine (7.0 mL, 88.0 mmol) to the reaction mixture by syringe and seal the 135 flask. Heat the flask to 100° C. with a heating mantle with stirring. After 21 hr., cool the reaction mixture to room temperature. Filter off the solids and concentrate the filtrate to an oily residue. Purify the residue by flash chromatography (800 g silica gel), eluting with heptane:MTBE (85:15). Recover the title compound as a colorless solid (4.07 g, 69% yield): MS (ES+) m/z 375 (M⁺+1); ¹H NMR (300 MHz, CDCl₃) δ 7.22 (m, 1H), 6.87 (m, 1H), 3.78-3.65 (m, 5H), 3.49-3.40 (m, 2H), 3.16 (m, 2H), 2.96 (m, 2H).

### 7-Chloro-6-(2,2,2-trifluoroethylamino)-2,3,4,5-tetrahydro-1H-benzo[d]azepine [1]

Add 5N NaOH (6 mL) to a solution of 7-chloro-3-(2,2,2-trifluoroacetyl)-6-(2,2,2-trifluoroethylamino)-2,3,4,5-tetrahydro-1H-benzo[d]azepine (above, 3.97 g, 10.59 mmol) in ethanol (20 mL) and stir the resulting solution for 30 min. at 23° C. Remove the solvent under vacuum and dissolve the residue in CH₂Cl₂. Wash the CH₂Cl₂ solution sequentially with water (20 mL), saturated aqueous NaCl (20 mL), water (20 mL), and finally saturated aqueous NaCl (50 mL). Dry the CH₂Cl₂ layer over Na₂SO₄ and evaporate the solvent to yield the title compound as an oil (2.84 g of the free base).

### EXAMPLE 2

### 7-Chloro-6-(2,2,2-trifluoroethylamino)-2,3,4,5-tetrahydro-1H-benzo[d]azepine Succinic Acid Salt

Dissolve 7-chloro-6-(2,2,2-trifluoroethylamino)-2,3,4,5-tetrahydro-1H-benzo[d]azepine free base (Example 1, 2.84 g, 10.19 mmol) in ethanol (15 mL) and treat with an ethanol solution of succinic acid (1.20 g, 10.19 mmol). Remove the solvent under vacuum to yield the title compound as a colorless solid (3.94 g, 97%). MS (ES+) m/z 279; ¹H NMR (300 MHz, DMSO-d₆), δ 7.18 (d, 1H), 6.88 (d, 1H), 4.92 (t, 1H), 3.68 (m, 2H), 2.91-3.08 (m, 8H), 2.28 (s, 4H). Alternatively, a more thermodynamically stable polymorph of the succinate salt may be obtained as follows: Dissolve 7-chloro-6-(2,2,2-trifluoroethylamino)-2,3,4,5-tetrahydro-1H-benzo[d]azepine (free base, 155.3 g, 0.548 mol) in isopropanol (1.72 L) and heat to 50° C. Slurry succinic acid (64.74 g, 0.548 mol) in isopropanol (1.37 L) and heat to 50° C. to give a solution. Add seed crystals to the solution of 7-chloro-6-(2,2,2-trifluoroethylamino)-2,3,4,5-tetrahydro-1H-benzo[d]azepine, then add the succinic acid solution at 50° C. over 2 min. It is typically observed that the exothermic reaction increases the reaction temperature to about 55° C. and a solid starts forming within 1-2 min. Allow the solution to cool over 1.5 hr. to about 38° C. Cool the solution further in an ice water bath to <5° C. and hold for 30 min. Filter off the solid, wash with isopropanol (300 mL, ˜5° C.), and dry in a vacuum oven at 45° C. to obtain the title compound as the form II polymorph (210.3 g, 96.7% yield). Differential scanning calorimetry: Onset Peak=159.5° C., Maximum Peak=161.0° C., Heat of Fusion 105.2 J/g.

The seed crystals of the thermodynamically more stable polymorph are obtained by the following equilibration study: dissolve 7-chloro-6-(2,2,2-trifluoroethylamino)-2,3,4,5-tetrahydro-1H-benzo[d]azepine (free base, 200 mg, 0.717 mmol) in isopropanol (3 mL) by heating to reflux (82° C.). Dissolve succinic acid (84 mg, 0.717 mmol) by heating in isopropanol (1 mL). Add the succinic acid solution to the refluxing 7-chloro-6-(2,2,2-trifluoroethylamino)-2,3,4,5-tetrahydro-1H-benzo[d]azepine solution and allow the resulting solution to cool. Crystallization occurs at approximately 40° C. and the resulting suspension is then heated at 50° C. for 66 hr. Cool the suspension of crystals to ambient temperature, filter, and dry to give seed crystals of 7-chloro-6-(2,2,2-trifluoroethylamino)-2,3,4,5-tetrahydro-1H-benzo[d]azepine succinic acid salt (230 mg, 81% yield): Differential scanning calorimetry: single peak at 160.9° C.

### EXAMPLE 3

### 7-Chloro-6-(2,2,2-trifluoroethylamino)-2,3,4,5-tetrahydro-1H-benzo[d]azepine mesylate Salt

Add methanesulfonic acid (46 μl, 0.71 mmol) to a 23° C. solution of 7-chloro-6-(2,2,2-trifluoroethylamino)-2,3,4,5-tetrahydro-1H-benzo[d]azepine (Example 1, 200 mg, 0.71 mmol) and isopropanol (4 mL). Cool the resulting suspension of crystals in an ice bath, filter, rinse with cold isopropanol (1 mL) and dry to give the title compound (227 mg, 85% yield). ¹H NMR (300 MHz, DMSO-d₆) δ 8.77 (s, 2H), 7.19 (d, 1H), 6.89 (d, 1H), 4.99 (t, 1H), 3.68 (m, 2H), 3.2-2.99 (m, 8H), 2.28 (s, 3H).

The compound of the present invention is relatively selective for the 5-HT_{2C} receptor. The compound of the present invention is particularly relatively selective for the 5-HT_{2C} receptor in comparison to other 5-HT receptor subtypes and specifically the 5-HT_{2A} and 5-HT_{2B} receptors. This selectivity is demonstrated in the following agonist activity assays and receptor binding assays.

Agonist Activity Assays (G Alpha g-GTPγ[³⁵S] Binding Assays)

The 5-HT₂ receptors are functionally coupled to specific G-proteins. Agonist activation of 5-HT₂ G-protein-coupled receptors results in the release of GDP from the α-subunit (G alpha q or G alpha i) of the G-protein and the subsequent binding of GTP. The binding of the stable analog GTPγ[³⁵S] is an indicator of receptor activation (i.e. agonist activity).

The G alpha q-GTPγ[³⁵S] binding assay is used to determine the in vitro potency (EC₅₀) and maximal efficacy (Eₘₐₓ, normalized to the 5-HT response) of a test compound at the 5-HT_{2A}, 5-HT_{2B}, and 5-HT_{2C} receptors. The area under the dose response curve (AUC) is also determined for each receptor subtype and used to measure the test compound's selectivity for the 5-HT_{2C} receptor over the 5-HT_{2A} and 5-HT_{2B} receptors, expressed as Selectivity Ratios (AUC 2C/2A and AUC 2C/2B, respectively). The Selectivity Ratios allow the assessment of selectivity based on both potency and efficacy. A selectivity measure that incorporates both potency and efficacy at the 5-HT_{2C} receptor, as compared to the 5-HT_{2A} and 5-HT_{2B}.receptors, is considered important due to the adverse events associated with 5-HT_{2A} and 5-HT_{2B} agonist activity (see introduction).

Membrane Preparation: Grow AV12 cells stably transfected with the human 5-HT_{2A}, 5-HT_{2B}, or 5-HT_{2C} receptors in suspension, harvest by centrifugation, wash the cell pellet with phosphate buffered saline, pH 7.4, pellet the cells again, remove the supernatant, freeze the cell pellet on dry ice and store at −70° C. Thaw stock cell pellet and resuspend in 50 mM Tris, pH 7.4, aliquot into 1-2 mL volumes and refreeze at −70° C. for subsequent assays (5-HT_{2A} and 5-HT_{2C} transfected cells: about 6×10⁸ cells per aliquot; 5-HT_{2B} cells: about 7.5×10⁸ cells per aliquot).

On the day of assay, thaw membranes, wash the membranes with assay buffer (50 mM Tris-HCl (pH 7.4), 10 mM MgCl₂, 100 mM NaCl, and 0.2 mM EDTA), resuspend in assay buffer and incubate for 10 min. at 37° C. to hydrolyze any residual endogenous 5-HT. Wash the membranes again with assay buffer, and resuspend in assay buffer at a concentration to provide aliquots of about 14×10⁶ cell equivalents per well (typically about 1-2×10⁶ cell equivalents for assays with 5-HT_{2A} or 5-HT_{2C} receptor assays, and about 3-4×10⁶ cell equivalents for assays with 5-HT_{2B} receptor assays). Homogenize the cells with a tissue grinder and use the homogenate directly in the assay as described below.

G alpha q-GTPγ[³⁵S] Binding Assays: The immunoadsorption scintillation proximity assay (ISPA) of [³⁵S]-GTPγS binding to G alpha q is modified from published conditions (DeLapp et al, JPET 289 (1999) 946-955). Dissolve test compounds in DMSO and dilute in assay buffer to provide a range of concentrations to generate a concentration response curve. In wells of a 96 well microtiter plate, mix diluted test compound, GDP (0.1 μM final concentration), and [³⁵S]-GTPγS (between 0.5 and 1.0 nM final concentration) Add an aliquot of membranes to the incubation mixture and mix the plates to initiate agonist stimulation of the nucleotide exchange (200 μl final volume). Incubate the microtiter plates for 30 min. at room temperature. Quench the incubation with IGEPAL® CA-630 detergent (0.27% final concentration). Add affinity purified polyclonal rabbit anti-G alpha q antibody (about 1-2 μg per well), and anti-rabbit Ig scintillation proximity assay beads (Amersham; about 1.25 mg per well; 290 μl final volume). Seal the plates and incubate the mixture for 3 hr. at room temperature. Centrifuge the microtiter plates briefly to pellet beads. Quantitate the GTPγ[³⁵] binding by microtiter plate scintillation spectrometry (Wallac Trilux MicroBeta™ scintillation counter).

Data Analysis: For each concentration response curve for a test compound at a given receptor, analyze the data with GraphPad Prism™ software (v3.02; GraphPad Software, San Diego, Calif.) running on a personal computer with MicroSoft Windows OS®, using nonlinear regression analysis curve fitting to determine the EC₅₀ and Eₘₐₓ (normalized to 5-HT control curves). Determine the Area Under the agonist concentration-response Curve (AUC) with GraphPad Prism™ by the trapezoidal method.

To calculate the Selectivity Ratios, first, determine the AUC for the test compound for each receptor subtype as described above. Second, normalize the AUC's at each receptor subtype relative to the AUC determined for 5-HT at that receptor. The normalized AUC for a test compound at a given receptor is therefore expressed as a percentage of the AUC determined for 5-HT at that receptor. For example:

$$5 {HT}_{2 A} Normalized AUC = a = \frac{\begin{matrix}( {AUC}_{test compound} at \\ 5 {HT}_{2 A} receptor )\end{matrix}}{\begin{matrix}( {AUC}_{5 \text{-} HT} at \\ 5 {HT}_{2 A} receptor )\end{matrix}} \times 100 \%$$

$$5 {HT}_{2 B} Normalized AUC = b = \frac{\begin{matrix}( {AUC}_{test compound} at \\ 5 {HT}_{2 B} receptor )\end{matrix}}{\begin{matrix}( {AUC}_{5 \text{-} HT} at \\ 5 {HT}_{2 B} receptor )\end{matrix}} \times 100 \%$$

$$5 {HT}_{2 C} Normalized AUC = c = \frac{\begin{matrix}( {AUC}_{test compound} at \\ 5 {HT}_{2 C} receptor )\end{matrix}}{\begin{matrix}( {AUC}_{5 \text{-} HT} 5 \text{-} HT at \\ 5 {HT}_{2 C} receptor )\end{matrix}} \times 100 \%$$

Third, calculate the Selectivity Ratios for the test compound as follows:
Selectivity Ratio for 5-HT_{2C} receptor/5-HT_{2A} receptor (AUC 2C/2A)=c/a
Selectivity Ratio for 5-HT_{2C} receptor/5-HT_{2B} receptor (AUC 2C/2B)=c/b

For reference purposes, the AUC 2C/2A and AUC 2C/2B for 5-HT are 1.0 and 1.0, respectively. Likewise, the ratios for mCPP (meta-chlorophenylpiperazine) are 2.1 and 2.1 respectively.

The compound of the present invention was tested in the G alpha q-GTPγ[³⁵S] assays for the 5-HT_{2A}, 5-HT_{2C}, and 5-HT_{2C} receptors essentially as described above and was surprisingly found to be a highly potent and selective agonist of the 5-HT_{2C} receptor. (See Table 1.)

TABLE 1
G alpha q-GTPγ[³⁵S] Agonist Activity Assays for
7-Chloro-6-(2,2,2-trifluoroethylamino)-2,3,4,5-tetrahydro-1H-benzo[d]azepine
succinic acid salt (Ex. 2)

| 5HT_{2A} | 5HT_{2A} | 5HT_{2B} | 5HT_{2B} | 5HT_{2C} | 5HT_{2C} | AUC | AUC |
|---|---|---|---|---|---|---|---|
| EC₅₀ (nM) | Eₘₐₓ | EC₅₀ (nM) | Eₘₐₓ | EC₅₀ (nM) | Eₘₐₓ | 2C/2A | 2C/2B |
| 696 ± 139 | 70.8 ± 7.2 | 119 ± 34 | 33.9 ± 1.0 | 11.8 ± 2.8 | 105.8 ± 2.8 | 2.2 | 3.8 |

(Error expressed is ± standard error of the mean)

Ligand Binding Assays

The ligand binding affinity of the compound of the present invention to the 5-HT_{2C} receptor subtype is measured essentially as described by Wainscott (Wainscott, et al., Journal of Pharmacology and Experimental Therapeutics, 276:720-727 (1996)). Data is analyzed by nonlinear regression analysis on the concentration response curves using the four parameter logistic equation described by DeLean (DeLean, et al., Molecular Pharmacology, 21, 5-16 (1982)). IC₅₀ values are converted to Kᵢ values using the Cheng-Prusoff equation (Cheng, et al., Biochem. Pharmacol., 22, 3099-3108 (1973)).

The compound of the present invention (Example 2) was tested essentially as described above and was found to have surprisingly excellent affinity for the 5-HT_{2C} receptor.

Affinities for other receptor subtypes can readily be determined by slight modification of the above described radioligand receptor binding assay using cells transfected with the desired receptor in place of cells transfected with the 5-HT_{2C} receptor subtype and using an appropriate radioligand. The binding affinities for the compound of the present invention for a variety of receptors were determined in such assays and the compound was found to have surprisingly higher affinity for the 5-HT_{2C} receptor. Affinity for the 5-HT_{2C} receptor was significantly higher than for other 5-HT receptor subtypes, and notably higher than the 5-HT_{2A} and 5-HT_{2B} receptor subtypes. IC50's for the compound of the present invention for the alpha 1 and alpha 2 adrenergic receptors and for D1 and D2 dopaminergic receptors were all found to be greater than 3000 nM.

Rat Feeding Assays

The ability of the compound of the present invention to treat obesity is demonstrated by testing in acute and chronic rat feeding assays.

Animals: Obtain male Long-Evans rats (Harlan Sprague-Dawley, Indianapolis, Ind.) that are approximately one hundred-days old and have been maintained on a calorie rich diet since weaning (TD 95217, 40% calories from fat; Teklad, Madison, Wis.). House the rats individually with a 12 hr.:12 hr. light:dark cycle (lights on from about 22:00 hr. to about 10:00 hr.) and maintain rats on the same diet (TD 95217) with free access to water, for about 1-2 weeks to acclimate the rats to the environment. Dose rats orally with vehicle (10% acacia with 0.15% saccharin in water) once daily for at least 1 day (typically 1-2 days) to acclimate the rats to the procedures. Randomize the rats into groups so each group has similar mean body weights.

Calorimetric Acute Feeding Assay: At approximately 8:00 hr. on the day of assay, weigh each rat and transfer to individual chambers of an open circuit calorimetry system (Oxymax, Columbus Instruments International Corporation; Columbus, Ohio), with free access to food (pre-weighed) and water, and begin measuring VO₂ and VCO₂. At approximately 10:00 hr., dose rats orally with vehicle or test compound, return them to their calorimetry chambers, and continue measuring VO₂ and VCO₂ at regular time intervals (approximately hourly). At approximately 8:00 hr. the following day, measure rat body weight and the remaining food, assuming the difference in weight of food is equal to the mass of food consumed. Calculate the 24 hr. energy expenditure (EE) and respiratory quotient (RQ) essentially as described in Chen, Y. and Heiman, M. L., Regulatory Peptide, 92:113-119 (2000). EE during light photoperiod is indicative of the resting metabolic rate and RQ is indicative of the fuel source the animal utilizes (pure carbohydrate metabolism gives an RQ of about 1.0, pure fat metabolism gives an RQ of about 0.7, mixed carbohydrate and fat metabolism gives intermediate values for RQ).

Calculate EE as the product of calorific value (CV) and VO₂ per body weight (kg); where CV=3.815+1.232*RQ, and RQ is the ratio of CO₂ produced (VCO₂) to O₂ consumed (VO₂). Caloric intake is calculated as (mass of 24 hr. food intake in grams)×(physiological fuel value of the diet in kilocalorie/g) per kg of body weight.

Acute Feeding Assay with a selective 5-HT_{2C} receptor antagonist: The above calorimetric acute feeding assay is conducted with the following modifications. Open circuit calorimetry systems are not used and only the 24 hr. periodic food intake and body weight are measured. Three groups of rats are used with the first group receiving a subcutaneous dose of saline (0.5 mL) about 15 minutes prior to the oral dose of vehicle, the second group receiving a subcutaneous dose of saline (0.5 mL) about 15 minutes prior to the oral dose of test compound in vehicle, and the third group receiving a subcutaneous injection of a selective 5-HT_{2C} receptor antagonist, 6-chloro-5-methyl-N-(2-(2-methylpyridin-3-yl-oxy)pyridin-5-yl)aminocarbonyl)-2,3-dihydroindole (3 mg/Kg, in 35% cyclodextrin, 0.5 mL), about 15 min. prior to the oral dose of test compound in vehicle.

Chronic Feeding Assay: At between approximately 8:00 hr. and 10:00 hr. on day one of the assay, weigh and orally dose each rat with vehicle or test compound and return the animal to its home cage, with free access to food (pre-weighed) and water. For each of days 2-15, at between approximately 8:00 hr. and 10:00 hr., measure rat body weight and the weight of food consumed in the last 24 hr. period, and administer daily oral dose of test compound or vehicle. On days −2 and 15 measure total fat mass and lean mass by nuclear magnetic resonance (NMR) using an EchoMRI™ system (Echo Medical Systems, Houston Tex.). (See Frank C. Tinsley, Gersh Z. Taicher, and Mark L. Heiman, “Evaluation of a New Quantitative Magnetic Resonance (QMR) Method for Mouse Whole Body Composition Analysis”, Obesity Research, submitted May 1, 2003.)

The compound of the present invention (Example 2) was tested in acute and chronic feeding assays essentially as described above. In the acute assays, the compound of the present invention was found to significantly reduce 24 hr. food intake, which effect was blocked by pre-administration of the 5-HT_{2C} receptor antagonist. The compound also dose-dependently reduced RQ without significantly changing the energy expenditure during the light photoperiod. Thus the compound reduced caloric intake and increased the proportion of fuel deriving from fat utilization, without significantly changing the rat's resting metabolic rate. In the chronic assay, the compound of the present invention was found to significantly decrease cumulative food intake and cumulative body weight change in a dose-dependent manner compared to control animals. The decrease in body weight was due to loss of adipose tissue while lean body mass was not changed.

The ability of the 5-HT_{2C} receptor agonist of the present invention to treat obsessive/compulsive disorder is demonstrated by testing in a variety of in vivo assays as follows:

Marble Burying Assay

Marble burying in mice has been used to model anxiety disorders including obsessive-compulsive disorders (OCD) due to ethological study of the behavior (e.g. Gyertyan I. “Analysis of the marble burying response: Marbles serve to measure digging rather than evoke burying”, Behavioural Pharmacology 6: 24-31, (1995)) and due to the pharmacological effects of clinical standards (c.f., Njung′E K. Handley S L. “Evaluation of marble-burying behavior as a model of anxiety”, Pharmacology, Biochemistry & Behavior. 38: 63-67, (1991)); Borsini F., Podhorna J., and Marazziti, D. “Do animal models of anxiety predict anxiolytic effects of antidepressants?”, Psychopharmacology 163: 121-141, (2002)). Thus, drugs used in the treatment of generalized anxiety in humans (e.g. benzodiazepines) as well as compounds used to treat OCD (e.g. SSRIs like fluoxetine) decrease burying.

House experimentally-naïve male, NIH Swiss mice (Harlan Sprague-Dawley, Indianapolis, Ind.) weighing between 28-35 g in groups of 12 for at least three days prior to testing in a vivarium with 12 hr. light and dark cycles. Conduct experiments during the light cycle in a dimly lit experimental testing room. Dose mice with vehicle or compound and, after a specified pretreatment interval (generally 30 min.), place each mouse individually on a rotorod (Ugo Basile 7650) operating at a speed of 6 revolutions/min. and observe for falling. After 2 min. on the rotorod, place the mice individually in a 17×28×12 cm high plastic tub with 5 mm sawdust shavings on the floor that are covered with 20 blue marbles (1.5 cm diameter) placed in the center. After 30 min., count the number of marbles buried (⅔ covered with sawdust). Assess the compound's effect on marble burying with Dunnett's test and the effect on rotorod performance by Fisher's exact test.

Clinically effective standard compounds suppress marble burying at doses that are devoid of motor-impairing effects as measured on the rotorod. The in vivo efficacy of 5HT_{2C} compounds at the 5HT_{2C} receptor is confirmed by the prevention of effects of the 5HT_{2C} agonists on marble burying by co-administration of the 5HT_{2C} receptor antagonist, 6-chloro-5-methyl-N-(2-(2-methylpyridin-3-yl-oxy)pyridin-5-yl)aminocarbonyl)-2,3-dihydroindole.

The compound of the present invention (Example 2) was assayed in the marble burying assay essentially as described and surprisingly found to reduce burying behavior in the test mice. The reduction of burying behavior was blocked by co-administration of the 5-HT_{2C} antagonist. In contrast to the compound of the present invention, the anxiolytic compound chlordiazepoxide and the antipsychotic compound chlorpromazine decreased marble burying only at doses that also disrupt rotorod performance.

Nestlet Shredding

Mice naturally will construct nests of material available in their living environment. Since this behavior is obsessive in nature, it has been used to model OCD (Mia Li, Denise Morrow and Jeffrey M. Witkin, “Decreases in nestlet shredding of mice by serotonin uptake inhibitors: comparison with marble burying”, Psychopharmacology, submitted Jul. 14, 2003). House experimentally-naïve male, NIH Swiss mice (Harlan Sprague-Dawley, Indianapolis, Ind.) weighing between 28-35 g in groups of 12 for at least three days prior to testing in a vivarium with a 12 hr. light/dark cycle. Conduct experiments during the light cycle in an experimental room with normal overhead fluorescent lighting. Dose mice with vehicle or test compound and after a specified pretreatment interval (generally 30 min.), place the mice individually in a 17×28×12 cm high plastic tub with about 5 mm sawdust shavings on the floor along with a pre-weighed multi-ply gauze pad (51 mm square). After 30 min., weigh the remainder of the gauze pad not removed by the mouse. Determine the weight of the gauze used for nestlet construction by subtraction. Compare the results for test compound treated mice to the results for vehicle control treated mice with Dunnett's test.

Clinically effective OCD treatment standard compounds suppress nestlet shredding at doses that are devoid of motor-impairing effects as measured by the rotorod test. The in vivo efficacy of 5HT_{2C} compounds at the 5HT_{2C} receptor was confirmed by the prevention of effects of the 5HT_{2C} agonists on nestlet shredding by co-administration of the 5HT_{2C} receptor antagonist, 6-chloro-5-methyl-N-(2-(2-methylpyridin-3-yl-oxy)pyridin-5-yl)aminocarbonyl)-2,3-dihydroindole.

The compound of the present invention (Example 2) was assayed essentially as described above and surprisingly found to suppress nestlet shredding at doses that were devoid of motor-impairing effects as measured by the rotorod test.

In contrast to the compound of the present invention, the anxiolytic chlordiazepoxide and the psychomotor stimulant d-amphetamine decrease nestlet shredding only at doses that produce motoric side effects (depression or stimulation, respectively).

Schedule-Induced Polydipsia

Food-deprived rats exposed to intermittent presentations of food will drink amounts of water that are far in excess of their normal daily intake and in excess of their intake when given all of their food at one time (Falk J L. “Production of polydipsia in normal rats by an intermittent food schedule”, Science 133: 195-196, (1961)). This excessive behavior is persistent and has been used to model OCD.

Maintain Wistar rats on a food restricted diet (to maintain 85% free feeding weight), but with free access to water. Train the rats in a behavioral testing chamber to press a lever to receive a food pellet under a fixed interval schedule, such that the rats are rewarded with a 45 mg food pellet the first time they press a lever after a 120 second interval has elapsed. The fixed interval is then reset to 120 seconds and the process repeated. Thus, during a 90 min. test session, the rats can earn a maximum of 45 pellets. The behavioral chamber is also equipped with a water bottle that is weighed before and after the session to determine the amount of water consumed.

Administer test compounds on Tuesdays and Fridays. Determine control day performances on Thursdays. Administer compounds either orally at 60 min. before the beginning of a test session, or subcutaneously at 20 min. before the beginning of a test session. Compare the rates of lever pressing and water consumption for each animal's performance during sessions after test compound treatment with that animal's performance during control sessions, expressed as a percent of the control rate. Average the individual percent of control rates for each dose and calculate the standard error of the mean.

Clinically effective OCD treatment standard compounds (e.g. chlomipramine, fluoxetine) suppress schedule-induced polydipsia without producing notable changes in motor patterns, food intake, or behavior the following day. The in vivo efficacy of 5HT_{2C} compounds at the 5HT_{2C} receptor was confirmed by the prevention of effects of the 5HT_{2C} agonists on excessive drinking by co-administration of the 5HT_{2C} receptor antagonist, 6-chloro-5-methyl-N-(2-(2-methylpyridin-3-yl-oxy)pyridin-5-yl)aminocarbonyl)-2,3-dihydroindole.

The compound of the present invention (Example 2) was assayed in the schedule-induced polydipsia assay essentially as described above and surprisingly found to suppress schedule-induced polydipsia without producing notable changes in motor patterns, food intake, or behavior the following day. The behavior suppression was blocked by co-administration of the 5-HT_{2C} antagonist.

In contrast to the compound of the present invention, the psychomotor stimulant d-amphetamine decreases excessive drinking only at behaviorally stimulating doses and these effects are not prevented by the 5HT_{2C} receptor antagonist.

Four-Day Rat Toxicology Study

Female Fischer 344 rats, 9 to 11 weeks of age, are housed individually with ad libitum access to food and water, and maintained at room temperature. Test compound or vehicle is administered by gavage in 10% Acacia, 0.05% Dow Corning Antifoam 1510-US, in purified water to test rats (n=3, 10 ml/kg), once daily for 4 days. Daily clinical observations, body-weight, and food consumption are recorded. Test rats are then fasted 4-15 hr. prior to necropsy. Isoflurane is used to anesthetize the rats and blood (about 0.6 mL) is retro-orbitally collected into each of two sample tubes, one sample tube with EDTA and one sample tube without EDTA, for each rat. Prepare serum and plasma samples for standard hematology and clinical chemistry measurements. Euthanize test animals by carbon dioxide asphyxiation. Remove the kidneys, liver, heart, spleen, adrenal, thymus, brain, and periuterine adipose tissue and record their weights. Remove the lung, stomach, duodenum, jejunum, ileum, diaphragm, and bone marrow, and dissect out the cerebellum, cerebrum, and brain stem. Fix the kidney, liver, heart, lung, spleen, adrenal, thymus; stomach, duodenum, jejunum, ileum, diaphragm, bone marrow, cerebellum, cerebrum, and brain stem in 10% neutral buffered formalin and process to slides for histological evaluation using standard hematoxylin and eosin staining procedures.

The compound of the present invention (Example 2) was assayed in a four-day rat toxicology study essentially as described above. The compound had a NOAEL (No Adverse Effect Level) of at least 50 mg/Kg in this assay.

While it is possible to administer a compound employed in the methods of this invention directly without any formulation, the compound is usually administered in the form of pharmaceutical compositions comprising a pharmaceutically acceptable excipient and a compound of formula I or a pharmaceutically acceptable salt thereof. These compositions can be administered by a variety of routes including oral, rectal, transdermal, subcutaneous, intravenous, intramuscular, and intranasal. The compound employed in the methods of this invention is effective as both injectable and oral compositions. Such compositions are prepared in a manner well known in the pharmaceutical art. See, e.g. REMINGTON'S PHARMACEUTICAL SCIENCES, (16th ed. 1980).

In making the compositions employed in the present invention the active ingredient is usually mixed with at least one excipient, diluted by at least one excipient, or enclosed within such a carrier which can be in the form of a capsule, sachet, paper or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing for example up to 10% by weight of the active compound, soft and hard gelatin capsules, suppositories, sterile injectable solutions, and sterile packaged powders.

In preparing a formulation, it may be necessary to mill the compound to provide the appropriate particle size prior to combining with the other ingredients. If the active compound is substantially insoluble, it ordinarily is milled to a particle size of less than 200 mesh. If the active compound is substantially water soluble, the particle size is normally adjusted by milling to provide a substantially uniform distribution in the formulation, e.g. about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxybenzoates; sweetening agents; and flavoring agents. The compositions of the invention can be formulated so as to provide quick, sustained or delayed release of the active ingredient after administration to the patient by employing procedures known in the art.

The compositions are preferably formulated in a unit dosage form, each dosage containing from about 0.05 to about 100 mg, more usually about 1.0 to about 30 mg, of the active ingredient. The term “unit dosage form” refers to physically discrete units suitable as unitary dosages for human subjects and other mammals, each unit containing a predetermined quantity of active material calculated to produce the desired therapeutic effect, in association with a suitable pharmaceutical excipient.

The compounds are generally effective over a wide dosage range. For examples, dosages per day normally fall within the range of about 0.01 to about 30 mg/kg. In the treatment of adult humans, the range of about 0.1 to about 15 mg/kg/day, in single or divided dose, is especially preferred. However, it will be understood that the amount of the compound actually administered will be determined by a physician, in the light of the relevant circumstances, including the condition to be treated, the chosen route of administration, the actual compound or compounds administered, the age, weight, and response of the individual patient, and the severity of the patient's symptoms, and therefore the above dosage ranges are not intended to limit the scope of the invention in any way. In some instances dosage levels below the lower limit of the aforesaid range may be more than adequate, while in other cases still larger doses may be employed.

Another preferred formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. Sees e.g., U.S. Pat. No. 5,023,252, issued Jun. 11, 1991, herein incorporated by reference. Such patches may be constructed for continuous, pulsatile, or on demand delivery of pharmaceutical agents.

Under some circumstances, it will be desirable or necessary to introduce the pharmaceutical composition to the brain, either directly or indirectly. Direct techniques usually involve placement of a drug delivery catheter into the host's ventricular system to bypass the blood-brain barrier. One such implantable delivery system, used for the transport of biological factors to specific anatomical regions of the body, is described in U.S. Pat. No. 5,011,472, issued Apr. 30, 1991, which is herein incorporated by reference.

Indirect techniques, which are generally preferred, usually involve formulating the compositions to provide for drug latentiation by the conversion of hydrophilic drugs into lipid-soluble drugs or prodrugs. Latentiation is generally achieved through blocking of the hydroxy, carbonyl, sulfate, and primary amine groups present on the drug to render the drug more lipid soluble and amenable to transportation across the blood-brain barrier. Alternatively, the delivery of hydrophilic drugs may be enhanced by intra-arterial infusion of hypertonic solutions which can transiently open the blood-brain barrier.

The type of formulation employed for the administration of the compounds employed in the methods of the present invention may be dictated by the particular compound employed, the type of pharmacokinetic profile desired from the route of administration, and the state of the patient.

1. A compound of formula I: 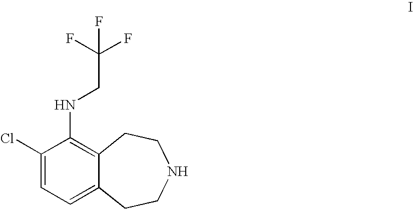
or a pharmaceutically acceptable salt thereof.

2. A pharmaceutical composition comprising a compound of claim 1 as an active ingredient in association with a pharmaceutically acceptable carrier, diluent or excipient.

3.-4. (canceled)

5. A method for the treatment of obesity in mammals, comprising administering to a mammal in need of such treatment an effective amount of a compound of claim 1.

6. The method of claim 5, where the mammal is human.

7. A method for the treatment of obsessive/compulsive disorder in mammals, comprising administering to a mammal in need of such treatment an effective amount of a compound of claim 1.

8. The method of claim 7, where the mammal is human.

9. A method for the treatment of depression in mammals, comprising administering to a mammal in need of such treatment an effective amount of a compound of claim 1.

10. The method of claim 9, where the mammal is human.

11. A method for the treatment of anxiety in mammals, comprising administering to a mammal in need of such treatment an effective amount of a compound of claim 1.

12. The method of claim 11, where the mammal is human.

13.-32. (canceled)
